(19) United States
(12) Patent Application Publication
(10) Pub. No.: US 2011/0160158 A1
(43) Pub. Date: Jun. 30, 2011

(54) METHODS OF TREATMENT OF BIPOLAR DISORDER

(75) Inventor: David R. Jacoby, Charlestown, MA (US)

(73) Assignee: REPLIGEN CORPORATION, Waltham, MA (US)

(21) Appl. No.: 12/997,296

(22) PCT Filed: Jun. 12, 2009
(86) PCT No.: PCT/US2009/047203

§ 371 Date: Mar. 14, 2011

Related U.S. Application Data
(60) Provisional application No. 61/061,061, filed on Jun. 12, 2008.

(51) Int. Cl. A61K 31/7072; A61P 25/18

(52) U.S. Cl. 514/50

(57) ABSTRACT

A method of treating an individual diagnosed with bipolar disorder, comprises determining the number of manic episodes and/or depressive episodes experienced by an individual exhibiting one or more symptoms of bipolar disorder (e.g., bipolar disorder I); and if the number of manic episodes and/or the number of depressive episodes is each 2 or greater (e.g., 3 or greater, 4 or greater, 5 or greater, 6 or greater, 10 or greater, or 15 or greater), administering to the individual an effective amount of a uridine composition.

### CROSS-REFERENCE TO RELATED APPLICATION

This application claims priority to U.S. Application Ser. No. 61/061,061, filed on Jun. 12, 2008, the entire contents of which are incorporated by reference herein.

### TECHNICAL FIELD

This invention relates to the treatment of bipolar disorder.

### BACKGROUND

Bipolar disorder, also known as manic-depressive illness, is a brain disorder characterized by periods of excitability (mania) alternating with periods of depression. About 5.7 million American adults or about 2.6 percent of the population age 18 and older in any given year, have bipolar disorder.

### SUMMARY

The invention is based, in part, on the discovery that individuals diagnosed with one or more symptoms of bipolar disorder who have a history of two or more episodes of mania (e.g., three or more, four or more, five or more, six or more, ten or more, or 15 or more) and/or two or more episodes of depression (e.g., three or more, four or more, five or more, six or more, ten or more, or 15 or more) derive a greater benefit from treatment with one or more pyrimidines, such as uridine, than individuals with fewer episodes of mania and/or depression.

Thus, the invention features methods of treating an individual diagnosed with bipolar disorder, that include: determining the number of manic episodes and/or depressive episodes experienced by an individual exhibiting one or more symptoms of bipolar disorder (e.g., bipolar I disorder or bipolar II disorder); and if the number of manic episodes (e.g., manic episodes and/or hypomanic episodes) or the number of depressive episodes (e.g., major depressive episodes) is 2 or greater (e.g., 3 or greater, 4 or greater, 5 or greater, 6 or greater, 7 or greater, 8 or greater, 9 or greater, 10 or greater, 12 or greater, or 15 or greater), administering to the individual an effective amount of a uridine composition. In some embodiments, if the number of manic episodes is fewer than 2 (e.g., fewer than 3, 4, 5, 6, 7, 8, 9, 10, 12, or 15), a uridine composition is not administered to the individual.

In a further aspect, the invention features methods of treating an individual diagnosed with bipolar disorder that include: determining the number of manic episodes experienced by an individual exhibiting one or more symptoms of bipolar disorder (e.g., bipolar I disorder or bipolar II disorder); determining the number of depressive episodes (e.g., major depressive episodes) experienced by the individual; and if the number of manic episodes (e.g., manic episodes and/or hypomanic episodes) is 2 or greater (e.g., 3 or greater, 4 or greater, 5 or greater, 6 or greater, 7 or greater, 8 or greater, 9 or greater, 10 or greater, 12 or greater, or 15 or greater) and the number of depressive episodes (e.g., major depressive episodes) is 2 or greater (e.g., 3 or greater, 4 or greater, 5 or greater, 6 or greater, 7 or greater, 8 or greater, 9 or greater, 10 or greater, 12 or greater, or 15 or greater), administering to the individual an effective amount of a uridine composition. In some embodiments, the individual is administered an effective amount of a uridine composition if the number of manic episodes is 2 or greater and the number of depressive episodes is 5 or greater. In some embodiments, if the number of manic episodes is fewer than 2 (e.g., fewer than 3, 4, 5, 6, 7, 8, 9, 10, 12, or 15) and/or the number of depressive episodes is fewer than 2 (e.g., fewer than 3, 4, 5, 6, 7, 8, 9, 10, or 15), a uridine composition is not administered to the individual.

In other aspects, the invention features use of a uridine composition in the preparation of a medicament for the treatment of bipolar disorder (e.g., bipolar I disorder or bipolar II disorder) with a history of two or more (e.g., 3 or more, 4 or more, 5 or more, 6 or more, 7 or more, 8 or more, 9 or more, 10 or more, 12 or more, or 15 or more) episodes of mania (e.g., mania and/or hypomania) or depression (e.g., major depression); use of a uridine composition for the treatment of bipolar disorder (e.g., bipolar I disorder or bipolar II disorder) with a history of two or more (e.g., 3 or more, 4 or more, 5 or more, 6 or more, 7 or more, 8 or more, 9 or more, 10 or more, 12 or more, or 15 or more) episodes of mania (e.g., mania and/or hypomania) or depression (e.g., major depression); use of a uridine composition in the preparation of a medicament for the treatment of bipolar disorder (e.g., bipolar I disorder or bipolar II disorder) with a history of two or more (e.g., 3 or more, 4 or more, 5 or more, 6 or more, 7 or more, 8 or more, 9 or more, 10 or more, 12 or more, or 15 or more) episodes of mania (e.g., mania and/or hypomania) and two or more (e.g., 3 or more, 4 or more, 5 or more, 6 or more, 7 or more, 8 or more, 9 or more, 10 or more, 12 or more, or 15 or more) episodes of depression (e.g., major depression); use of a uridine composition for the treatment of bipolar disorder (e.g., bipolar I disorder or bipolar II disorder) with a history of two or more (e.g., 3 or more, 4 or more, 5 or more, 6 or more, 7 or more, 8 or more, 9 or more, 10 or more, 12 or more, or 15 or more) episodes of mania (e.g., mania and/or hypomania) and two or more (e.g., 3 or more, 4 or more, 5 or more, 6 or more, 7 or more, 8 or more, 9 or more, 10 or more, 12 or more, or 15 or more) episodes of depression (e.g., major depression); use of a uridine composition in the preparation of a medicament for the treatment of bipolar disorder (e.g., bipolar I disorder or bipolar II disorder) with a history of two or more episodes of mania (e.g., mania and/or hypomania) and five or more episodes of depression (e.g., major depression); and use of a uridine composition for the treatment of bipolar disorder (e.g., bipolar I disorder or bipolar II disorder) with a history of two or more episodes of mania (e.g., mania and/or hypomania) and five or more episodes of depression (e.g., major depression).

In another aspect, the invention features methods of selecting an individual for treatment of bipolar disorder that include: evaluating whether an individual exhibits one or more symptoms of bipolar disorder (e.g., bipolar I disorder or bipolar II disorder); determining the number of manic episodes (e.g., manic episodes and/or hypomanic episodes) and depressive episodes (e.g., major depressive episodes) experienced by the individual; and selecting the individual for treatment with a uridine composition if the number of manic episodes or depressive episodes is 2 or greater (e.g., 3 or greater, 4 or greater, 5 or greater, 6 or greater, 7 or greater, 8 or greater, 9 or greater, 10 or greater, 12 or greater, or 15 or greater). In some embodiments, methods include selecting the individual for treatment with a uridine composition only if the number of manic episodes or depressive episodes is 2 or greater (e.g., 3 or greater, 4 or greater, 5 or greater, 6 or greater, 7 or greater, 8 or greater, 9 or greater, 10 or greater, 12 or greater, or 15 or greater).

In another aspect, the invention features methods of selecting an individual for treatment of bipolar disorder that include: evaluating whether an individual exhibits one or more symptoms of bipolar disorder (e.g., bipolar I disorder or bipolar II disorder); determining the number of manic episodes (e.g., manic episodes and/or hypomanic episodes) experienced by the individual; determining the number of depressive episodes (e.g., major depressive episodes) experienced by the individual; and selecting the individual for treatment with a uridine composition if the number of manic episodes is 2 or greater (e.g., 3 or greater, 4 or greater, 5 or greater, 6 or greater, 7 or greater, 8 or greater, 9 or greater, 10 or greater, 12 or greater, or 15 or greater) and the number of depressive episodes is 2 or greater (e.g., 3 or greater, 4 or greater, 5 or greater, 6 or greater, 7 or greater, 8 or greater, 9 or greater, 10 or greater, 12 or greater, or 15 or greater). In some embodiments, the methods include selecting the individual for treatment with a uridine composition only if the number of manic episodes is 2 or greater (e.g., 3 or greater, 4 or greater, 5 or greater, 6 or greater, 7 or greater, 8 or greater, 9 or greater, 10 or greater, 12 or greater, or 15 or greater) and the number of depressive episodes is 2 or greater (e.g., 3 or greater, 4 or greater, 5 or greater, 6 or greater, 7 or greater, 8 or greater, 9 or greater, 10 or greater, 12 or greater, or 15 or greater). In some embodiments, the methods include selecting the individual for treatment with a uridine composition if (e.g., only if) the number of manic episodes is 2 or greater and the number of depressive episodes is 5 or greater.

In some embodiments of the above methods and uses, an effective amount of the uridine composition provides about 1 to 50 mg of a uridine composition/kg of body weight/day, e.g., about 3 to 10 mg, about 3 to 20 mg, about 5 to 15 mg, or about 7 to 10 mg of a uridine composition/kg of body weight/day.

In some embodiments of the above methods and uses, the individual is diagnosed under DSM-IV guidelines as having bipolar disorder and/or an effective amount of the uridine composition comprises between 250 and 2000 mg of uridine composition/day.

In some embodiments of the above methods and uses, the uridine composition is administered in two or three doses/day.

In some embodiments of the above methods and uses, the uridine composition is uridine, triacetyl uridine, uridine monophosphate, uridine diphosphate, or uridine triphosphate.

In some embodiments of the above methods and uses, the uridine composition is prepared in a form for oral administration.

In another aspect, the invention features kits that include a medicament that includes a uridine composition (e.g., uridine, triacetyl uridine, uridine monophosphate, uridine diphosphate, or uridine triphosphate) and instructions to administer an effective amount of the composition to an individual with bipolar disorder (e.g., bipolar I disorder or bipolar II disorder) if the individual has a history of two or more (e.g., 3 or more, 4 or more, 5 or more, 6 or more, 7 or more, 8 or more, 9 or more, 10 or more, 12 or more, or or more) episodes of mania (e.g., mania and/or hypomania) and/or two or more (e.g., 3 or more, 4 or more, 5 or more, 6 or more, 7 or more, 8 or more, 9 or more, 10 or more, 12 or more, or 15 or more) episodes of depression (e.g., major depression).

The invention also features methods of evaluating a candidate treatment for bipolar disorder (e.g., bipolar I disorder or bipolar II disorder) that include: obtaining a population of individuals that exhibit one or more symptoms of bipolar disorder; determining the total number of manic episodes (e.g., manic episodes and/or hypomanic episodes) and or depressive episodes (e.g., major depressive episodes) experienced by each individual; selecting a subpopulation of the population of individuals for whom the total number of manic episodes or depressive episodes is 2 or greater (e.g., 3 or greater, 4 or greater, 5 or greater, 6 or greater, 7 or greater, 8 or greater, 9 or greater, 10 or greater, 12 or greater, or 15 or greater); and administering a candidate treatment for bipolar disorder to the subpopulation. In some embodiments, the methods further include determining the effect of the candidate treatment to treat, reduce, or alleviate one or more symptoms of bipolar disorder in the individuals of the subpopulation. In some embodiments, the methods further include administering a placebo to some individuals of the subpopulation and assaying whether the candidate treatment is effective to treat, reduce, or alleviate one or more symptoms of bipolar disorder to a greater extent than the placebo does.

The invention also features methods of evaluating a candidate treatment for bipolar disorder (e.g., bipolar I disorder or bipolar II disorder) that include: obtaining a population of individuals that exhibit one or more symptoms of bipolar disorder; determining the total number of manic episodes (e.g., manic episodes and/or hypomanic episodes) experienced by each individual; determining the total number of depressive episodes (e.g., major depressive episodes) experienced by each individual; selecting a subpopulation of the population of individuals for whom the total number of manic episodes is 2 or greater (e.g., 3 or greater, 4 or greater, 5 or greater, 6 or greater, 7 or greater, 8 or greater, 9 or greater, 10 or greater, 12 or greater, or 15 or greater) and the total number of depressive episodes is 2 or greater (e.g., 3 or greater, 4 or greater, 5 or greater, 6 or greater, 7 or greater, 8 or greater, 9 or greater, 10 or greater, 12 or greater, or or greater); and administering a candidate treatment for bipolar disorder to the subpopulation. In some embodiments, a subpopulation of individuals is selected for whom the total number of manic episodes is 2 or greater and the total number of depressive episodes is 5 or greater. In some embodiments, the methods further include determining the effect of the candidate treatment to treat, reduce, or alleviate one or more symptoms of bipolar disorder in the individuals of the subpopulation. In some embodiments, the methods further include administering a placebo to some individuals of the subpopulation and assaying whether the candidate treatment is effective to treat, reduce, or alleviate one or more symptoms of bipolar disorder to a greater extent than the placebo does.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing the weekly change from baseline (S) of the average Montgomery Åsberg Depression Rating Scale (MADRS) scores for the placebo and uridine treated groups. The graph depicts scores for each time point in the treatment phase of the study, S depicts start date (baseline) and the numbers refer to the weeks on study (e.g., 6 is week 6 the final study assessment). The scores are shown as mean change of the MADRS from baseline (study day 1). Boxes represent the difference in the response; hatched boxes depict treatment effect that favors uridine, solid boxes depict treatment effect that favors placebo.

FIG. 2 is a graph depicting weekly values for the average Clinical Global Impression of Severity of Bipolar Disorder (CGI-C-BP) (Overall) for placebo and uridine groups for each evaluation. The boxes represent the magnitude of the difference between uridine and placebo.

FIG. 3 is a graph depicting the weekly values for the change from baseline of the average MADRS scores for subjects with a history of ≧15 episodes of depression.

FIG. 4 is a graph depicting the weekly values for the change from baseline of the average MADRS scores for subjects with a history of >15 episodes of depression.

FIG. 5 is a graph depicting the weekly values for the change from baseline of the average MADRS scores for subjects with a history of ≧10 episodes of depression (Day 43, p=0.18; Δ=5.8).

FIG. 6 is a graph depicting the weekly values for the change from baseline of the average MADRS scores for subjects with a history of <10 episodes of depression (Day 43, Δ=−1.7).

FIG. 7 is a graph depicting the weekly values for the change from baseline of the average MADRS scores for subjects with a history of ≧15 episodes of mania or hypomania.

FIG. 8 is a graph depicting the weekly values for the change from baseline of the average MADRS scores for subjects with a history of ≧2 episodes of mania or hypomania (Day 43, p=0.11; Δ=5.7).

FIG. 9 is a graph depicting the weekly values for the change from baseline of the average MADRS scores for subjects with a history of <2 episodes of mania or hypomania (Day 43, Δ=−8.2).

### DETAILED DESCRIPTION

The new methods and uses described herein are based on the surprising discovery that individuals diagnosed with bipolar disorder who have a history of two or more episodes of mania and/or two or more episodes of depression derive a greater benefit from treatment by the administration of one or more pyrimidines, such as uridine, prodrugs of uridine, and undine analogs, than individuals diagnosed with bipolar disorder who have a history of fewer episodes of mania and/or depression. Such individuals can be selected for treatment by the administration of an effective amount of a pyrimidine composition such as a uridine composition, for example, by oral or systemic intravenous administration. The new uses and methods are based on the results of a human clinical trial to determine the effectiveness of uridine for treatment of patients with bipolar disorder.

### Bipolar Disorder

Bipolar disorder is a chronic illness associated with substantial morbidity and mortality, ranking worldwide behind only unipolar depression and alcohol abuse among psychiatric illnesses for related disabilities and overall economic burden of illness. The lifetime financial burden of bipolar disorder in the United States is about $625,000 per patient (Begley et al., 2001, Pharmacoeconomics, 19:483-495). Lithium and anticonvulsants such as divalproex have substantially improved the prognosis of bipolar disorder (Bowden, 2000, J. Clin. Psychiatry, 61(Suppl 9):35-40). However, many individuals are unable to tolerate treatment-related side-effects, and have incomplete clinical responses, with relapse and recurrence common clinical problems (Hirschfeld et al., 2007, Psychopharmacol. Bull., 40:7-14).

The symptoms of bipolar disorder can be severe, and can result in emotional problems, poor job or school performance, and even suicide. The name “bipolar” comes from the patients' mood swings, which can alternate between the “poles” of mania (highs) and depression (lows). These mood swings can be quite dramatic, from overly “high” and/or irritable to sad and hopeless, and then back again, often with periods of normal mood in between, and severe changes in energy and behavior go along with these changes in mood. Bipolar disorder typically develops in late adolescence or early adulthood. However, some people have their first symptoms during childhood or late in life. This disorder is not always viewed as an illness, and people may suffer for years before proper diagnosis.

Bipolar disorder has been separated into two categories, Type I and Type II, and can be diagnosed following the guidelines in the Diagnostic and Statistical Manual of Mental Disorders (DSM) Fourth Edition, 1994 (American Psychiatric Association, 1400 K Street NW, Suite 1101, Washington, D.C. 20005-2403 USA). The fourth edition of these guidelines, DSM-IV, identifies the diagnostic features of Bipolar I Disorder as follows.

Bipolar I Disorder (DSM-IV, p. 350)

This disorder is a clinical course that is characterized by the occurrence of one or more Manic Episodes or Mixed Episodes. Often individuals have also had one or more Major Depressive Episodes. Episodes of Substance-Induced Mood Disorder (due to the direct effects of a medication, or other somatic treatments for depression, a drug of abuse, or toxin exposure) or of Mood Disorder Due to a General Medical Condition do not count toward a diagnosis of Bipolar I Disorder. In addition, the episodes are not better accounted for by Schizoaffective Disorder and are not superimposed on Schizophrenia, Schizophreniform Disorder, Delusional Disorder, or Psychotic Disorder Not Otherwise Specified.

The diagnostic features of Bipolar II Disorder are as follows.

S Bipolar II Disorder (DSM-IV, p. 359)

This disorder is a clinical course that is characterized by the occurrence of one or more Major Depressive Episodes accompanied by at least one Hypomanic Episode. Hypomanic Episodes should not be confused with the several days of euthymia that may follow remission of a Major Depressive Episode. Episodes of Substance-Induced Mood Disorder (due to the direct effects of a medication, or other somatic treatments for depression, a drug of abuse, or toxin exposure) or of Mood Disorder Due to a General Medical Condition do not count toward a diagnosis of Bipolar I Disorder. In addition, the episodes are not better accounted for by Schizoaffective Disorder and are not superimposed on Schizophrenia, Schizophreniform Disorder, Delusional Disorder, or Psychotic Disorder Not Otherwise Specified.

The following diagnostic criteria, also from the DSM-IV apply.

Criteria for Major Depressive Episode (DSM-IV, p. 327)

A. Five (or more) of the following symptoms have been present during the same 2-week period and represent a change from previous functioning; at least one of the symptoms is either (1) depressed mood or (2) loss of interest or pleasure. Symptoms that are clearly due to a general medical condition, or mood-incongruent delusions or hallucinations should not be included.
Depressed mood most of the day, nearly every day, as indicated by either subjective report (e.g., feels sad or empty) or observation made by others (e.g., appears tearful). Note: In children and adolescents, can be irritable mood.
Markedly diminished interest or pleasure in all, or almost all, activities most of the day, nearly every day (as indicated by either subjective account or observation made by others)
Significant weight loss when not dieting or weight gain (e.g., a change of more than 5% of body weight in a month), or decrease or increase in appetite nearly every day. Note: In children, consider failure to make expected weight gains.
Insomnia or hypersomnia nearly every day.—Psychomotor agitation or retardation nearly every day (observable by others, not merely subjective feelings of restlessness or being slowed down).
Fatigue or loss of energy nearly every day.—Feelings of worthlessness or excessive or inappropriate guilt (which may be delusional) nearly every day (not merely self-reproach or guilt about being sick).
Diminished ability to think or concentrate, or indecisiveness, nearly every day (either by subjective account or as observed by others).
Recurrent thoughts of death (not just fear of dying), recurrent suicidal ideation without a specific plan, or a suicide attempt or a specific plan for committing suicide.

B. The symptoms do not meet criteria for a Mixed Episode.

C. The symptoms cause clinically significant distress or impairment in social, occupational, or other important areas of functioning.

D. The symptoms are not due to the direct physiological effects of a substance (e.g., a drug of abuse, a medication) or a general medical condition (e.g., hypothyroidism).

E. The symptoms are not better accounted for by bereavement, i.e., after the loss of a loved one, the symptoms persist for longer than 2 months or are characterized by marked functional impairment, morbid preoccupation with worthlessness, suicidal ideation, psychotic symptoms, or psychomotor retardation.

Criteria for Manic Episode (DSM-IV, p. 332)

A. A distinct period of abnormally and persistently elevated, expansive, or irritable mood, lasting at least 1 week (or any duration if hospitalization is necessary).

B. During the period of mood disturbance, three (or more) of the following symptoms have persisted (four if the mood is only irritable) and have been present to a significant degree:
Inflated self-esteem or grandiosity.
Decreased need for sleep (e.g., feels rested after only 3 hours of sleep).
More talkative than usual or pressure to keep talking.
Flight of ideas or subjective experience that thoughts are racing.
Distractibility (i.e., attention too easily drawn to unimportant or irrelevant external stimuli).
Increase in goal-directed activity (either socially, at work or school, or sexually) or psychomotor agitation.
Excessive involvement in pleasurable activities that have a high potential for painful consequences (e.g., engaging in unrestrained buying sprees, sexual indiscretions, or foolish business investments).

C. The symptoms do not meet criteria for a Mixed Episode.

D. The mood disturbance is sufficiently severe to cause marked impairment in occupational functioning or in usual social activities or relationships with others, or to necessitate hospitalization to prevent harm to self or others, or there are psychotic features.

E. The symptoms are not due to the direct physiological effects of a substance (e.g., a drug of abuse, a medication, or other treatments) or a general medical condition (e.g., hyperthyroidism).

Manic-like episodes that are clearly caused by somatic antidepressant treatment (e.g., medication, electroconvulsive therapy, light therapy) should not count toward a diagnosis of Bipolar I Disorder.

Criteria for Mixed Episode (DSM-IV, p. 335)

A. The criteria are met both for a Manic Episode and for a Major Depressive Episode (except for duration) nearly every day during at least a 1-week period.

B. The mood disturbance is sufficiently severe to cause marked impairment in occupational functioning or in usual social activities or relationships with others, or to necessitate hospitalization to prevent harm to self or others, or there are psychotic features.

C. The symptoms are not due to the direct physiological effects of a substance (e.g., a drug of abuse, a medication, or other treatment) or a general medical condition (e.g., hyperthyroidism).

Criteria for Hypomanic Episode (DSM-IV, p. 338)

A. A distinct period of persistently elevated, expansive, or irritable mood, lasting throughout at least 4 days, that is clearly different from the usual nondepressed mood.

B. During the period of mood disturbance, three (or more) of the following symptoms have persisted (four if the mood is only irritable) and have been present to a significant degree:
Inflated self-esteem or grandiosity.
Decreased need for sleep (e.g., feels rested after only 3 hours of sleep).
More talkative than usual or pressure to keep talking.
Flight of ideas or subjective experience that thoughts are racing.
Distractibility (i.e., attention too easily drawn to unimportant or irrelevant external stimuli).
Increase in goal-directed activity (either socially, at work or school, or sexually) or psychomotor agitation.
Excessive involvement in pleasurable activities that have a high potential for painful consequences (e.g., engaging in unrestrained buying sprees, sexual indiscretions, or foolish business investments).

C. The episode is associated with an unequivocal change in functioning that is uncharacteristic of the person when not symptomatic.

D. The disturbance in mood and the change in functioning are observable by others.

E. The episode is not severe enough to cause marked impairment in social or occupational functioning, or to necessitate hospitalization, and there are no psychotic features.

F. The symptoms are not due to the direct physiological effects of a substance (e.g., a drug of abuse, a medication, or other treatment) or a general medical condition (e.g., hyperthyroidism).

Hypomanic-like episodes that are clearly caused by somatic antidepressant treatment (e.g., medication, electroconvulsive therapy, light therapy) should not count toward a diagnosis of Bipolar II Disorder.

The diagnosis of symptoms of bipolar disorders (e.g., mania, hypomania, and depression (e.g., major depression)) in an individual or one or more of a population of individuals can be determined by a person skilled in the art, e.g., a psychiatrist, psychologist, or clinician. Diagnostic rating scales can be used to aid in diagnosis of bipolar disorders and specific symptoms, e.g., MADRS (Montgomery Asberg Depression Rating Scale); CGI-BP-S (Clinical Global Impression of Severity of Bipolar Disorder); Hamilton Anxiety Scale (HAM-A); YMRS (Young Mania Rating Scale); and CGI-BP-C (Clinical Global Impression of Change in Bipolar Disorder).

MADRS is a clinical assessment of depression that has been used in many clinical studies of therapeutic intervention. The scale is a 10 item inventory of the core symptoms and cognitive features in which symptoms are rated on a 0-6 scale (Montgomery, 1979, Br. J. Psychiatry, 134:382-389). The scale is used to measure symptom severity for a specified interval. Total scores correlate highly with the Hamilton rating scale (HAM-D) and with training show a high inter-rater reliability (Williams, 2008, Br. J. Psychiatry, 192: 52-58).

The CGI-BP scale is a modification of the clinical global impression for use in bipolar disorder as a global assessment of patient severity and change of status (Spearing et al., 1997, Psychiatry Res., 73:159-171). The CGI-BP-S rates the severity of the illness on a 7 point scale. The CGI-BP-C rates the change in the severity of disease compared to baseline. This also uses a 7 point scale, anchored by a score of 4 which correlates to no change since the initiation of treatment. Lower scores correspond to improvement, higher to worsening.

The YMRS is an 11 item scale designed to measure core manic symptoms (Young et al., Br. J. Psychiatry, 133:429-435). A cumulative score of 15 or greater on the YMRS was considered evidence of a treatment emergent affective switch. The HAM-A scale is a measure of global anxiety, including cognitive and somatic symptoms.

### Uridine Compositions

A uridine composition is either purified uridine, a compound or product that contains uridine, a compound that increases the level of uridine in the patient, or a compound or molecule that mimics the biological function of uridine. Such a compound can be a uridine precursor or prodrug, which is processed, e.g., metabolized, degraded, or cleaved, in the body to form uridine. Such a compound can also be a uridine derivative, which includes uridine, and other molecules or compounds bound (e.g., covalently or non-covalently) to uridine, but that do not impair uridine's biological activity in patients with increased purine levels. Such compounds can also be uridine mimetics, such as other nucleotides or small molecules that have a sufficiently similar three-dimensional shape or electron configuration that the compound has at least 50 percent of the biological activity of uridine. Such compounds can also be drugs or other compounds that induce the body to produce uridine, or drugs or compounds that inhibit degradation or otherwise prolong the half-life of uridine in the body.

Uridine precursors or prodrugs include orotic acid, mono-, di- or tri-esters of uridine, including mono-, di-, and triacetyl uridine, and mono, di- or tri-phosphates of uridine including uridine monophosphate (UMP) undine diphosphate (UDP) and uridine triphosphate (UTP). Uridine mimetics include cytidine and mono-, di-, or tri-phosphates of cytidine including cytidine monophosphate, as well as mono-, di-, or tri-esters of cytidine including triacetyl cytidine. Deoxy-versions of these and other ribonucleosides may also be useful.

Uridine compositions also include encapsulated uridine, e.g., liposome- or polymer-encapsulated uridine. Uridine compositions also include uridine linked (e.g., covalently or non-covalently) to various antibodies, ligands, or other targeting and enveloping or shielding agents (e.g., albumin or dextrose), to allow the uridine to reach the target site (e.g., the central nervous system, muscle cells, or the peripheral nervous system) prior to being removed from the blood stream, e.g., by the kidneys and liver, and prior to being degraded.

Uridine salts or food products containing uridine that transform into uridine upon administration to a host such as human can also be used.

Useful uridine-containing compounds include, without limitation, any compound comprising uridine, UTP, UDP, or UMP.

Oral uridine-based compounds have been investigated in patients with hereditary orotic aciduria (Webster, D. R., “Hereditary Orotic Aciduria and Other Disorders of Pyrimidine Metabolism,” in The Metabolic and Molecular Bases of Inherited Disease (Vol II), C. R. Scriver et al., Eds. 1995, McGraw-Hill), adenyl succinate lyase deficiency (Salerno et al., 2000, Adv. Exp. Med. Biol., 486:75-78), autism and pervasive developmental delay (Page et al., 1997, Proc. Natl. Acad. Sci. USA, 94:11601-06; Page and Coleman, 1998, Adv. Exp. Med. Biol., 431:793-796; Page and Coleman, 2000, Biochim. Biophys. Acta, 1500:291-296; Page. and Moseley, 2002, Prog. Neuropsychopharm. Biol. Psychiat., 26:397-400), diabetic neuropathy (Gallai et al., 1992, Acta Neurol. Scand., 86:3-7), patients undergoing chemotherapy with fluorouracil (5-FU) (van Groeningen et al., 1991, J. Natl. Cancer Inst., 83:437-441), and retroviral therapy in patients with HIV (Banasch et al., 2006, AIDS, 20:1554-56). The largest experience has been in hereditary orotic aciduria where some patients have been treated for over twenty years at doses as high as 300 mg/kg/d. The side effects noted with high dose oral uridine were diarrhea and abdominal cramps, typically at doses greater than 240 mg/kg, which raised plasma uridine levels to 60-80 μM.

Intravenous (W) uridine has been used in the context of cancer chemotherapy at doses that have resulted in sustained elevations in plasma uridine, sometimes exceeding 2,000 μM (van Groeningen et al., 1986, Cancer Treat. Rep., 70:745-750; Leyva et al., 1984, Cancer Res., 44:5928-33). The main side effects seen with IV uridine were infusion site phlebitis and transient shivering and fever.

Triacetyluridine (TAU) is converted to uridine in the gut mucosa, therefore it is essentially a pro-drug of uridine. Studies with TAU have been performed in children with mitochondrial disease, patients undergoing chemotherapy with 5-fluorouracil (5-FU), and in patients with monopolar depression. In the studies of children with mitochondrial disease, doses of TAU at 2 g/m², three times daily were used for up to two years. Significant symptomatic improvement was noted, particularly in the frequency of seizures and in the accompanying renal tubular acidosis, and no adverse events were reported (Naviaux et al., “Clinical Experience with Uridine and triacetyluridine (PN401) Therapy of Mitochondrial Disease,” in Mitochondrial Dysfunction in Human Pathology. 1998. Melboune, Australia; Naviaux et al., “Correction of Renal Tubular Acidosis (RTA) in Mitochonrdrial Disease Patients Treated with Triacetyluridine (PN401),” in Society for Inherited Metabolic Disorders Annual Meeting. 1999. Atlanta, Ga.; Naviaux, R. K., “Pyrimidine Therapy of Mitochondrial Disease,” in NIH minisymposium. 2000). In the chemotherapy studies, patients received 6 g of TAU four times a day with no adverse effects (Kelsen et al., 1997, J. Clin. Oncol., 15:1511-17; Hidalgo et al., 2000, J. Clin. Oncol., 18:167-177).

### General Methods of Therapy

The new methods involve the administration of an effective amount of a pyrimidine composition, such as a uridine composition, or a candidate treatment for bipolar disorder to an individual diagnosed with one or more symptoms of bipolar disorder and a history of two or more episodes of mania and/or two or more episodes of depression. The uridine composition or candidate treatment can be formulated into a therapeutic composition and administered using a variety of known routes of administration, and in various dosage forms.

To formulate pharmaceutical grade therapeutic compositions, the uridine composition can be purified by standard methods, e.g., filtration, to remove contaminants, if present. The final compositions can be lyophilized and resuspended in sterile, deionized water before further compounding. The therapeutic compositions can be formulated as solutions, suspensions, suppositories, tablets, granules, powders, capsules, ointments, or creams. In the preparation of these compositions, at least one pharmaceutical excipient can be included. Examples of pharmaceutical excipients include solvents (e.g., water or physiological saline), solubilizing agents (e.g., polysorbates, or Cremophor EL7), agents for achieving isotonicity, preservatives, antioxidizing agents, lactose, crystalline cellulose, mannitol, maltose, calcium hydrogen phosphate, light silicic acid anhydride, calcium carbonate, binders (e.g., starch, polyvinylpyrrolidone, hydroxypropyl cellulose, ethyl cellulose, carboxy methyl cellulose, or gum arabic), lubricants (e.g., magnesium stearate, talc, or hardened oils), or stabilizers (e.g., lactose, mannitol, maltose, polysorbates, macrogols, or polyoxyethylene hardened castor oils). If desired, glycerin, dimethylacetamide, lactate, surfactant, or basic substances such as sodium hydroxide, ethylenediamine, ethanolamine, sodium bicarbonate, arginine, meglumine, or trisaminomethane can be added. Common disintegrants that can be included in the composition include croscarmellose sodium, crospovidone, gellan gum, and sodium starch glycolate.

When the pyrimidine composition, e.g., a uridine composition, is ingested, the excipient or carrier can be water, a flavored beverage such as a fruit juice, broth, carbonated beverage, milk, or milk shake.

Biodegradable polymers such as poly-D,L-lactide-co-glycolide or polyglycolide can be used as a bulk matrix if slow release of the composition is desired (see, e.g., U.S. Pat. Nos. 5,417,986, 4,675,381, and 4,450,150). Pharmaceutical preparations such as solutions, tablets, granules or capsules can be formed with these components. If the composition is to be administered orally, flavorings and/or colors can be added.

The new compositions can be administered via any appropriate route, e.g., intravenously, intraarterially, topically, transdermally, by injection, intraperitoneally, intrapleurally, orally, subcutaneously, intramuscularly, sublingually, nasally, by inhalation, intraepidermally, or rectally, using standard techniques.

Dosages administered in practicing the new methods will depend on factors including the specific uridine composition used and its concentration in the composition, the mode and frequency of administration, the age, weight, sex, and general health of the subject, and the severity of the manic and/or depressive symptoms. In general, the new compositions can be administered in amounts ranging between 1.0 mg and 200 mg of uridine per kilogram of body weight per day, e.g., 2, 3, 5, 10, 20, 50, or 100 mg/kg/day.

A typical dosage is between 3 and 100 mg/kg/day, e.g., which can be 0.25 to 7 grams (e.g., 0.25, 0.5, or 1 grams) per patient per day. Oral tablets of triacetyl uridine can be used. The daily dosage is administered on an ongoing basis until symptoms subside.

An exemplary oral administration form of uridine contains 500 mg of active substance and percent composition weight/weight: Uridine, 66.7%; Croscarmellose, 3.0%; Microcrystalline Cellulose, 10.0%; Maltodextrin, 8.0%; Lactose, 10.9%; Magnesium Stearate, 1.25%; and Silicon Dioxide, 0.2%.

Dosages can be administered with meals or once, twice, or more times per day to achieve the best relief of symptoms. The dosage should be adjusted to provide a reduction in symptoms. Once the proper dosage is determined, it can be easily maintained over time as required. Typically, 5 to 15 μM is the normal plasma concentration of uridine with a volume distribution around 0.634 liters/kg. Following administration of a uridine composition, e.g., triacetyl uridine, blood plasma levels of about 50 to 300 μM are in the typical therapeutic range.

Administration is repeated as necessary, as determined by one skilled in the art. By varying the amount of the composition or dosage, the administration protocol can be optimized based on the present disclosure to elicit a maximal improvement in symptoms of bipolar disorder. Physicians, pharmacologists, and other skilled artisans are able to determine the most therapeutically effective treatment regimen, which will vary from patient to patient. The potency of a specific composition and its duration of action can require administration on an infrequent basis, including administration in an implant made from a polymer that allows slow release of the uridine.

Skilled artisans are also aware that the treatment regimen must be commensurate with issues of safety and possible toxic effect produced by the uridine or other components in the compositions. Thus, before administering the above compositions to humans, toxicity testing can be conducted in animals, e.g., as described in Examples below. In an example of toxicity testing, the uridine compositions can be administered to mice via an oral or parenteral route with varying dosages of uridine in the composition, and the mice observed for signs of toxicity using standard techniques. Of course, if the uridine composition is pure uridine, long-term experience has shown that uridine has no known toxic effects at dosages of up to 1000 mg/kg/day. Higher dosages may cause mild diarrhea in some patients. See, e.g., Leyva et al., Cancer Res., 4:5928-5933 (1984)(high dose uridine used to rescue patients from 5-fluorouracil toxicity) and Webster et al., Chapter 55, pages 1799-1837, in “The Metabolic and Molecular Bases of Inherited Disease,” 7th Ed., Scriver et al. (eds.)(McGraw-Hill, Inc., New York, N.Y., 1995) (treatment of orotic aciduria with uridine, see, e.g., page 1815).

Combination with Other Therapeutics

The pyrimidine, e.g., uridine, compositions described herein can be administered as a monotherapy, as combinations of two or more different pyrimidines, e.g., uridine compositions (or uridine and cytidine compositions), or in combination with other compounds for the treatment of bipolar disorders. For example, the pyrimidine compositions can be administered in conjunction with lower doses of current treatments for bipolar disorder, including stimulants and antidepressants. For example, divalproex sodium (DEPAKOTE®) has been used to treat bipolar disorder. In particular examples, the pyrimidine compositions may be administered in combination with an antidepressant, anticonvulsant, antianxiety, antimanic, antipyschotic, antiobsessional, sedative, stimulant, or anti-hypertensive medication. Examples of these medications include, serotonin reuptake inhibitors, monoamine oxidase inhibitors, tricyclic antidepressants, dopamine agonists (e.g., bromocriptine, pergolide), bupropion, venlafaxine, nefazodone, benzodiazepine, trazodone, lithium (Li), risperidone, topiramate, lamotrigine, gabapentin, nimodipine, divalproex, quetiapine, divalproex, lamotrigine, carbamazepine, clozapine, olanzapine, topiramate, thyroid hormone (e.g., T3 or T4), Omega-3 fatty acids, calcium channel blockers (other than nimodipine), tiagabine, cholinesterase inhibitors, tamoxifen, and phenytoin.

### Kits

A uridine composition, e.g., uridine, triacetyl uridine, uridine monophosphate, uridine diphosphate, or uridine triphosphate, can be provided in a kit. The kit can include (a) the composition, e.g., a dosage form that includes the uridine composition, and (b) informational material, such as a label. The informational material can be descriptive, instructional, marketing, or other material that relates to the methods described herein and/or the use of the uridine composition for the methods described herein.

In one embodiment, the informational material can include instructions to administer the uridine composition in a suitable manner to perform the methods described herein, e.g., in a suitable dose, dosage form, or mode of administration (e.g., a dose, dosage form, or mode of administration described herein). In another embodiment, the informational material can include instructions to administer the uridine composition to a suitable subject, e.g., an individual with bipolar disorder with a history of two or more (e.g., 3 or more, 4 or more, 5 or more, 6 or more, 7 or more, 8 or more, 9 or more, 10 or more, 12 or more, or 15 or more) episodes of mania (e.g., mania and/or hypomania); an individual with bipolar disorder with a history of two or more (e.g., 3 or more, 4 or more, 5 or more, 6 or more, 7 or more, 8 or more, 9 or more, 10 or more, 12 or more, or 15 or more) episodes of depression (e.g., major depression); or an individual with bipolar disorder with a history of two or more (e.g., 3 or more, 4 or more, 5 or more, 6 or more, 7 or more, 8 or more, 9 or more, 10 or more, 12 or more, or 15 or more) episodes of mania (e.g., mania and/or hypomania) and/or two or more (e.g., 3 or more, 4 or more, 5 or more, 6 or more, 7 or more, 8 or more, 9 or more, 10 or more, 12 or more, or 15 or more) episodes of depression (e.g., major depression). In some embodiments, the informational material can include instructions not to administer the composition to a patient with fewer than 2 (e.g., fewer than 3, 4, 5, 6, 7, 8, 9, 10, 12, or 15) episodes of mania and/or fewer than 2 (e.g., fewer than 3, 4, 5, 6, 7, 8, 9, 10, 12, or 15) episodes of depression.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, electronic mail address, web address, or telephone number, where a user of the kit can obtain substantive information about the uridine composition and/or its use in the methods described herein. Of course, the informational material can also be provided in any combination of formats.

In addition to the uridine composition, the composition of the kit can include other ingredients, such as a solvent or buffer, a stabilizer, a preservative, a fragrance or other cosmetic ingredient, and/or a second agent for treating a condition or disorder described herein. Alternatively, the other ingredients can be included in the kit, but in different compositions or containers than the uridine composition. In such embodiments, the kit can include instructions for admixing the uridine composition and the other ingredients, or for using the agent together with the other ingredients. The uridine composition can be provided in any form, e.g., liquid, dried or lyophilized form. It is preferred that the agent be substantially pure and/or sterile. When the uridine composition is provided in a liquid solution, the liquid solution is typically an aqueous solution, e.g., a sterile aqueous solution. When the uridine composition is provided as a dried form, reconstitution generally is by the addition of a suitable solvent. The solvent, e.g., sterile water or buffer, can optionally be provided in the kit.

The kit can include one or more containers for the uridine composition. In some embodiments, the kit contains separate containers, dividers, or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agent. For example, the kit includes a plurality of syringes, ampules, foil packets, or blister packs, each containing a single unit dose of the agent. The containers of the kits can be air tight and/or waterproof.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device.

### Methods of Evaluation

Methods of evaluating a candidate treatment for bipolar disorder will typically include selecting a population of individuals for study, administering the candidate treatment to at least a portion of the individuals, and monitoring the response to the treatment of the portion administered the treatment. In some embodiments, the methods will also include administering a control treatment (e.g., a placebo) to at least a portion of the individuals, e.g., the portion not administered the candidate treatment.

Individuals can be selected such a study based on several negative and positive factors. For example, individuals can be selected who have been diagnosed with bipolar disorder and have a history of either or both of two or more (e.g., 3 or more, 4 or more, 5 or more, 6 or more, 7 or more, 8 or more, 9 or more, 10 or more, 12 or more, or 15 or more) episodes of mania (e.g., mania and/or hypomania) and/or two or more (e.g., 3 or more, 4 or more, 5 or more, 6 or more, 7 or more, 8 or more, 9 or more, 10 or more, 12 or more, or 15 or more) episodes of depression (e.g., major depression). In some embodiments, individuals with fewer than 2 (e.g., fewer than 3, 4, 5, 6, 7, 8, 9, 10, 12, or 15) episodes of mania and/or fewer than 2 (e.g., fewer than 3, 4, 5, 6, 7, 8, 9, 10, 12, or 15) episodes of depression are excluded from the study. Individuals can also be selected to be included in the study or excluded from the study based on, e.g., age, current symptoms, history of drug or substance dependence, known history of sensitivity to the treatment.

The response to the treatment can be monitored by standard methods of diagnosis and evaluation of bipolar disorder, e.g., MADRS (Montgomery Åsberg Depression Rating Scale); CGI-BP-S (Clinical Global Impression of Severity of Bipolar Disorder); Hamilton Anxiety Scale (HAM-A); YMRS (Young Mania Rating Scale); and CGI-BP-C (Clinical Global Impression of Change in Bipolar Disorder). Typically, the response of the individuals administered the candidate treatment is compared to the response of the individuals administered the control treatment to determine if there was an effect of the treatment, e.g., a statistically significant effect. The response can be monitored over a set period of time, e.g., two weeks, three weeks, four weeks, six weeks, eight weeks, ten weeks, twelve weeks, four months, five months, six months, or one year.

### Example

A six-week, double blind, placebo-controlled, dose-escalation study was performed to assess the safety and efficacy of uridine in bipolar I depression. Eight-four (84) patients of both sexes were enrolled at twelve sites, randomized on a 1:1 basis to receive either placebo or uridine. To decrease the probability of a false negative result, uridine or placebo were given as monotherapy, and no concurrent bipolar medications, including mood stabilizers, atypical antipsychotics, antidepressants, anticonvulsants or other psychotropic medications were allowed during the study. Patients taking such medications were tapered gradually during the 28 days prior to initiation of the trial. Patients were evaluated each week over the 6-week trial period, with an extensive intervention plan for any patient having exacerbation of bipolar or depressive symptoms. After the trial period, patients were eligible to initiate therapy for bipolar disease and a follow-up visit occurred at week ten.

Patients were selected that had a DSM-IV-TR diagnosis of Bipolar I Disorder, Most Recent Episode Depressed, as confirmed on the MINI; 18 to 65 years of age, inclusive; currently depressed, as measured by MADRS >20 at Screening and Day 1, with duration of current depressive episode of at least four weeks by Day 1. Informed consent was provided by all patients. Patients were excluded who had a manic/hypomanic/mixed episode as determined by the MINI evaluation at Screening and/or a Young Mania Rating Scale (YMRS) score of >12 at Screening and/or Day 1; dementia or any current Axis I diagnosis other than bipolar I requiring pharmacological treatments; a history of alcohol or substance dependence within six months of Day 1, or a history of alcohol or substance abuse within three months of Day 1; urine drug screen positive for amphetamines, cocaine metabolites, opiates and/or phencyclidine (PCP); an Axis II diagnosis that was likely to interfere with protocol compliance, initiation of or increase in psychotherapy within 4 weeks of Screening; psychotropic medication (excluding fluoxetine) within 24 hours of initiation of study drug on Day 1; fluoxetine within 2 weeks of initiation of study drug on Day 1; serious suicidal or homicidal risk as determined by the investigator and/or a score of >5 on the suicide item #10 of the MADRS at Screening and/or Day 1; history of sensitivity to any of the ingredients in the study drug; clinically significant abnormality in any screening laboratory results; clinically significant organic disease, including cardiovascular, endocrine, hepatic, pulmonary, neurologic, or renal disease, or any other medical condition, serious intercurrent illness, or extenuating circumstances that, in the opinion of the investigator, would interfere with the performance or interpretability of, or put the patient at risk from, the study procedures; women who were pregnant, breastfeeding, or refused to use adequate birth control; current seizure disorder; participation in an investigational drug study within twenty-eight days of Day 1; current psychotic episode; clozaril use and/or electroconvulsive therapy within six months of Day 1; failure of three or more adequate trials of standard therapies for depression during the current episode; or current episode of depression longer than one year.

Patients could be withdrawn from the study for any of the following reasons: a score of 5 or greater on item #10 of the MADRS; a 30% increase on Total MADRS Score from Day 1; psychotic episode based on DSM-IV-TR criteria; a score of >15 on any administration of the YMRS and/or a patient's meeting DSM-IV-TR criteria for a manic, mixed or hypomanic episode; use of any psychotropic medications other than benzodiazepines (equivalent to a total daily dose of 2 mg lorazepam) and sedatives/hypnotics (equivalent to a total daily dose of 10 mg zolpidem); use of 1) benzodiazepines for more than 14 days or 2) sedatives/hypnotics for more than 14 days during the treatment phase of the study; initiation of or increase in psychotherapy during the treatment phase; occurrence of a clinically unacceptable adverse event; violation of the study protocol; patient declines further study participation; in the investigator's judgment, it was in the patient's best interest (i.e. investigator becomes aware of suicidal ideation/plan).

Subjects in the uridine treatment group were given either 2 g per day (low dose) or 4 g per day (high dose) of uridine. Subjects in the placebo group were given tablets of identical size and appearance with identical dosing instructions.

A dose of 1 g orally twice daily (2 g/day) was chosen to initiate treatment, with an increase to 2 g orally twice daily (4 g/day) in the absence of clinical improvement. On Day 1, all patients received a single dose of either placebo or uridine (2 g). This loading dose was done in order to compare pharmacokinetics of uridine at study start and end. Beginning on Day 2, patients received either placebo and uridine at a dose of 2 g/day administered as 1 g orally twice daily (2 tablets twice daily) for one week. At the Day 8 visit, the dose was increased to 4 g/day administered as 2 g orally twice daily (4 tablets twice daily) if there had been no serious untoward drug-related adverse events, and if the subject had less than a 25% improvement on the MADRS from baseline (Day 1). Patients that remained on 2 g/day were increased to 4 g/day at a subsequent visit if the improvement from baseline on the MADRS was less than 25%. Likewise, subjects having adverse events at the high dose were decreased to the low dose at the discretion of the investigator. On Day 43, all patients received a single dose of either placebo or uridine administered as 2 g orally twice daily (4 tablets twice daily), regardless of dose regimen. This was the patients' last dose of study drug.

The MINI is a brief psychiatric inventory that was administered to the subject at Screening in order to confirm the clinical diagnosis of bipolar I disorder, most recent episode depressed.

MADRS was administered at Screening and at every subsequent visit. This metric was the measure of depression severity during the trial. It was also a formalized measure of suicide risk (item #10). A response of 4 to item 10 required a signed risk assessment plan to be implemented and a response of 5 was to result in study discontinuation and a therapeutic intervention.

YMRS was administered at Screening and at every subsequent visit. This metric was the measure of mania severity during the study. A total score of ≧15 at any administration of the YMRS was to result in study termination and therapeutic intervention.

In order to reduce both inter-rater and intra-rater variability of clinical outcome measures, and to ensure the clinical quality control of the raters, the study used an interactive patient interview to confirm that the current symptomatology was being captured accurately. The computer interview was voluntary and the data was not used in any efficacy analysis. If there were discordances in the patient's reported symptoms and the clinical outcome measures recorded for that visit, the raters were queried and the scores were justified. This clinical quality control was performed by a centralized auditor, Concordant Rater Systems.

Clinical investigators were chosen with significant experience using the neuropsychiatric metrics employed by this study. To minimize the inter- and intra-rater variability, raters were re-trained on evaluation methods at the outset of the trial. During the course of the trial, rater evaluations were corroborated by remote site monitoring performed by Concordant Rating Systems (Lexington, Mass.). At each scheduled visit, patients were asked about their bipolar depression symptoms, using a personal computer interview. This data was employed as a rater quality control tool, to ensure that symptoms were being captured during the assessment. The rating assessment was intended to maintain the standardization of multi-center evaluation techniques throughout the course of the study.

83 patients were included in the trials to determine efficacy of uridine to treat bipolar disorder. The primary efficacy outcome was the mean MADRS assessment of the uridine group compared to placebo using a mixed effects repeat measures model. The difference of the mean MADRS scores for the uridine group in comparison to placebo was statistically significant, for both total scores (p=0.011) and percent change from baseline (p=0.014). Table 1 shows the mean MADRS score and percent change from baseline for uridine and placebo at each study evaluation. The baseline MADRS assessment was similar for the two groups, which was expected because it was a randomization criterion. This study was not adequately powered to detect differences between groups at single time points. Despite this, there are trends towards treatment difference at week 3 (p=0.10) and week 4 (p=0.06).

The week 6 end of study assessments demonstrated a strong change from baseline for the RG2417-treated group, with a mean change of 17.6 points (58.1%). The final assessment 4 weeks after study drug administration was completed showed no rebound in the MADRS assessments for either group. The MERM change in mean MADRS score of the RG2417 compared to placebo was used to calculate an effect size of 0.43 for difference with baseline adjusted as a covariate. This effect size is indicative of a treatment benefit.

TABLE 1
Average MADRS scores for treatment groups
at each weekly assessment

| Time | n¹ | Placebo | Δ (%)² | n¹ | RG2417 | Δ (%)² | P³ |
|---|---|---|---|---|---|---|---|
| Baseline⁴ | 44 | 30.68 | 0 | 39 | 30.28 | 0 | 0.75 |
| Wk 1 | 41 | 22.20 | −27.6 | 37 | 22.46 | −25.8 | 0.89 |
| Wk 2 | 37 | 21.38 | −30.3 | 36 | 18.81 | −37.9 | 0.23 |
| Wk 3 | 33 | 19.76 | −35.6 | 29 | 15.90 | −52.5 | 0.10 |
| Wk 4 | 30 | 18.40 | −40.0 | 30 | 13.93 | −54.0 | 0.06 |
| Wk 5 | 27 | 15.96 | −48.0 | 29 | 14.97 | −50.6 | 0.69 |
| Wk 6 | 25 | 16.13 | −47.4 | 29 | 12.69 | −58.1 | 0.26 |
| Wk 10⁵ | 21 | 19.05 | −37.9 | 23 | 15.91 | −47.5 | 0.41 |

MERM⁶ p (score difference) = 0.011;
p (% change from baseline) = 0.014
¹The number of patients evaluated at each time point
²The change from baseline evaluation (Study Day 1), stated as a percentage of the baseline score
³Pairwise comparisons of the scores for each treatment group for that time point.
⁴The baseline evaluation was at Study Day 1
⁵The treatment phase of the study concluded at week 6. A follow-up visit at week 10 a month after treatment has concluded.
⁶Mixed effects repeat measure from day 1 to day 43

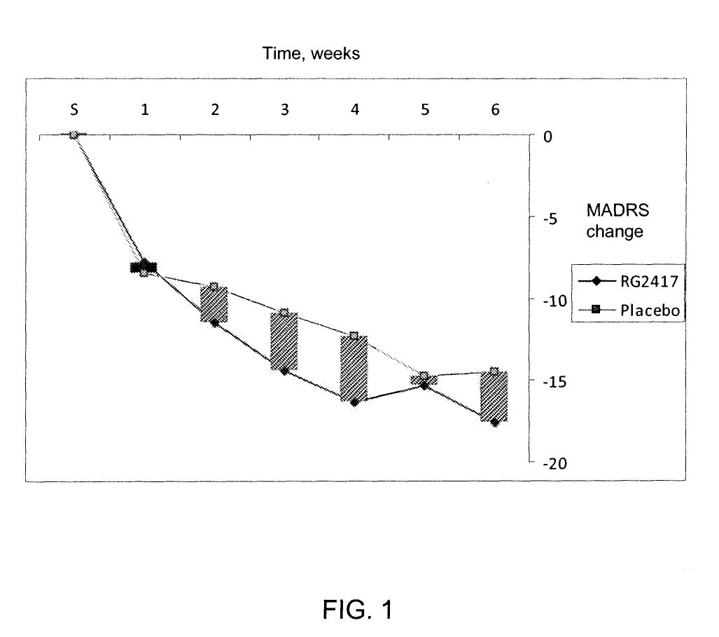

The change from baseline (study day 1) in the mean MADRS assessment for RG2417 and placebo is illustrated in FIG. 1. A clear treatment difference was observed over the course of the study, with treatment benefit designated by the hatched boxes at each time point. The true difference in the mean, i.e., the difference in response corrected for baseline [(baseline_{Uridine}−week 6_{Uridine})−(baseline_{Placebo}−week 6_{Placebo})] MADRS scores favored uridine from week 2, with an average difference of 3.2 points from week 3 to week 6. There was a deviation in the treatment difference at a single time point, week 5, in which there was a mild decrement in the treatment response and a substantial increase in the placebo response. Indeed, the study was characterized by an improvement in the placebo scores for weeks 4, 5, and 6. In prior published successful efficacy studies in bipolar depression, MADRS placebo responses of 10-12 points are typical (Tohen et al., 2003, Arch. Gen. Psychiatry, 60:1079-88; Sysko et al., 2007, J. Clin. Psychiatry, 68:1213-17).

The CGI-BP is a clinical impression scale that has been adapted for bipolar disorder and is comprised of an overall score, a depression score and a mania score. The CGI in this study used the severity of disease (CGI-BP-S) at study day 1 as the anchor point and the change from baseline (CGI-BP-C) in subsequent evaluations. Each assessment was intended to measure the change from the study start. The severity of disease (baseline) was similar between groups (Uridine=4.48; Placebo=4.68, n.s.), which are similar in magnitude to published efficacy trials in bipolar I depression (Brown et al., 2006, J. Clin. Psychiatry, 67:1025-33; Calabrese et al., 1999, J. Clin. Psychiatry, 60:79-88; Thase et al., 2006, J. Clin. Psychiatry, 26:600-609; Tohen et al., 2003, Arch. Gen. Psychiatry, 60:1079-88; Goldberg et al., 2007, Am. J. Psychiatry, 164:1348-55; Nierenberg et al., 2006, Am. J. Psychiatry, 163:210-216). The mean CGI-BP-C (overall) and CGI-BP-C (depression) for RG2417 in comparison to placebo is shown in Table 2, below. The primary outcome for CGI is the difference between RG2417 and placebo using MERM analysis of the treatment period. There was a significant difference to the change in CGI associated with RG2417 treatment (p=0.044).

Similar to the MADRS evaluations, the greatest difference in treatment occurred at weeks 3 and 4, with some erosion in the separation of the two groups at weeks 5 and 6, predominantly due to increase in placebo response. Comparison between groups of the overall CGI-BP-C showed no single time point with a significant difference between treatment and placebo, however there was a trend towards significance at week 3 (p=0.07). Analysis of the CGI-BP-C depression scale showed a more robust difference between uridine and placebo, achieving significance at week 3 (p=0.03) and week 4 (p=0.05).

TABLE 2
Average clinical global impression of change
for bipolar disorder (CGI-C-BP)

| | CGI-C-BP (Overall)¹ | | | CGI-C-BP (Depression)² | | |
|---|---|---|---|---|---|---|
| Time | Placebo | RG2417 | p | Placebo | RG2417 | P |
| Wk 1 | 3.20 | 3.19 | 0.98 | 3.10 | 3.05 | 0.86 |
| Wk 2 | 3.05 | 2.92 | 0.58 | 3.03 | 2.72 | 0.23 |
| Wk 3 | 2.97 | 2.45 | 0.07 | 2.94 | 2.31 | 0.03 |
| Wk 4 | 2.70 | 2.33 | 0.21 | 2.67 | 2.10 | 0.05 |
| Wk 5 | 2.44 | 2.31 | 0.64 | 2.41 | 2.14 | 0.34 |
| Wk 6 | 2.43 | 2.21 | 0.52 | 2.43 | 2.03 | 0.27 |
| p-value³ | 0.044 | | | 0.029 | | |

¹CGI-C-BP is scored as the change from baseline (Study Day 1) on a 7-point scale: 1- very much improved, 2- much improved, 3- minimally improved, 4- no change, 5- minimally worse, 6- much worse, 7- very much worse.
²CGI-C-BP for depression is the specific evaluation of the effect of treatment on the clinical symptoms of depression.
³Mixed effects repeat measure from study day 1 to day 43.

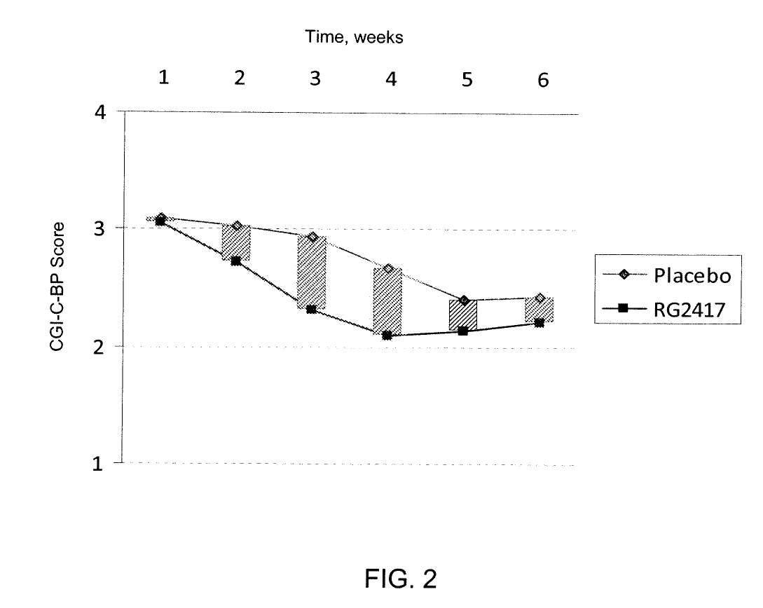

The mean CGI-BP-C over the study period for uridine and placebo is shown also in FIG. 2. The magnitude of the difference between groups is shown by the boxes at each time point. The magnitude of response appeared to be attained by week 4 for the uridine treatment and remained stable over the remainder of the study. The weeks 5 and 6 CGI-C assessments again revealed an increase in the placebo response, which reduced the overall difference in treatment effect at the later time points. A difficulty in the methodology of the CGI-C-BP was that the anchor point is at study start and so sequential assessments are incrementally more removed from the rating benchmark.

The mean change in the YMRS assessment of uridine treated subjects in comparison to placebo was also observed. The baseline evaluation at study day 1 showed a significant difference (p=0.01) with the uridine group having a higher initial mean score. Although the difference at baseline makes comparative interpretation difficult, it is apparent that uridine treatment did not exacerbate the mean YMRS, as there were no visits on treatment in which the mean values exceeded the baseline value, in contrast to placebo in which 2 of the 6 mean weekly YMRS scores were mildly increased. Analysis using MERM while on treatment showed a significant decrease in the YMRS from baseline for uridine-treated subjects in comparison to placebo (p=0.025).

The effect on anxiety in uridine-treated bipolar I depression subjects in comparison to placebo was evaluated using the mean HAM-A scores for each group. There was no single time point for which the mean HAM-A score was significantly different between groups. Likewise, there was no difference between groups over the treatment period using MERM analysis.

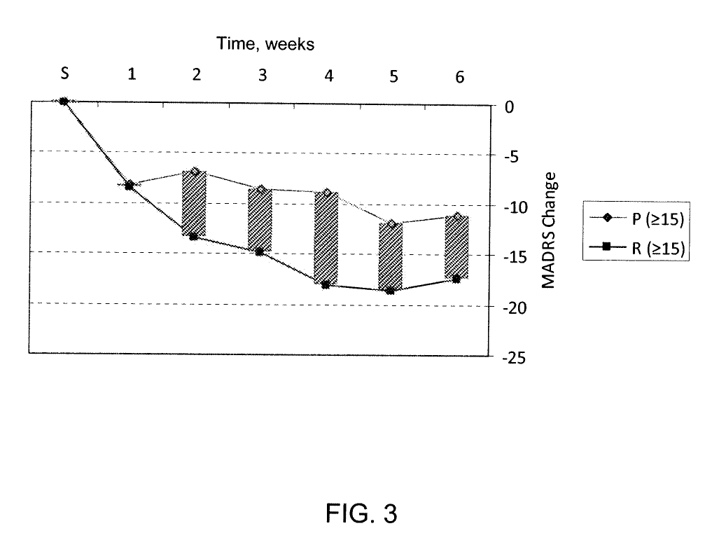

The response of bipolar I subjects with a history of 15 or more episodes of depression were analyzed by comparison of the mean MADRS score to placebo during the trial. This criterion was intended to analyze the response in individuals with a history of a more severe disease course. The overall response to treatment and the difference from placebo was greater for subjects with a greater history of depression (FIG. 3). The true difference in mean between groups is 6.35 at week 6, compared to 3.04 for the total score. The major component of this difference is in the placebo arm, in which the scores are less robust compared to the total group. For example, at study end (week 6) the placebo response in this subset was 36% (change from baseline), in comparison to 47% for the total efficacy cohort. The treatment response at study end was 58% for both the subset and the total group. The effect size ranges from 0.53 to 1.09 for weeks 3 to 6 in this subset. Clinical assessments were significant different between groups at week 4 and trend towards significance at week 3 and 5.

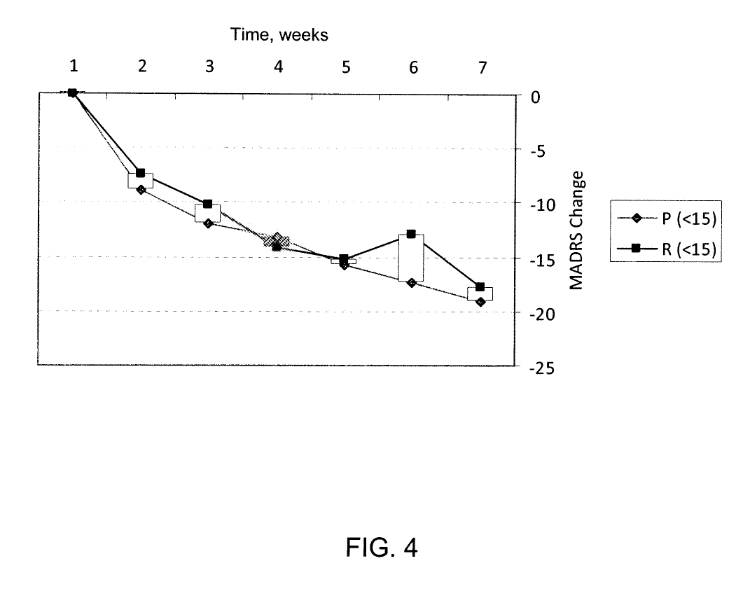

Subjects with a history of illness with fewer depressive episodes were also analyzed for efficacy response. This subset consisted of the remaining 49 subjects with <15 historic episodes of depression, of which the majority (34) had just 1-5 total episodes. This subset had no appreciable difference in mean MADRS scores between treatment and placebo groups (FIG. 4). The placebo response was extremely large, accounting for a 62% change from baseline at study end. The uridine group had a 58% improvement from baseline which was similar to the change in the overall scores.

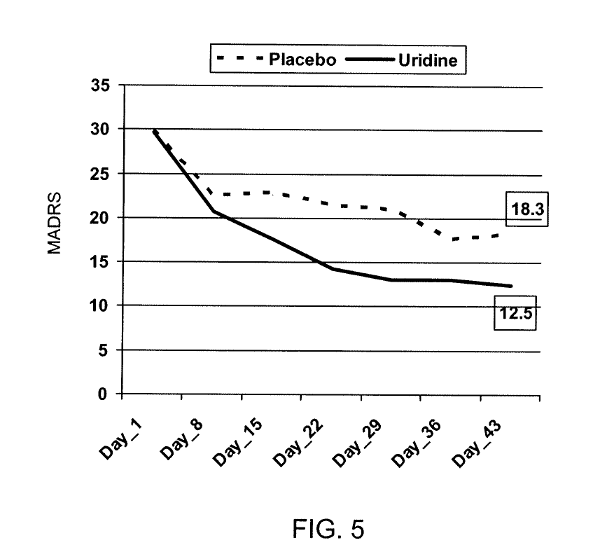

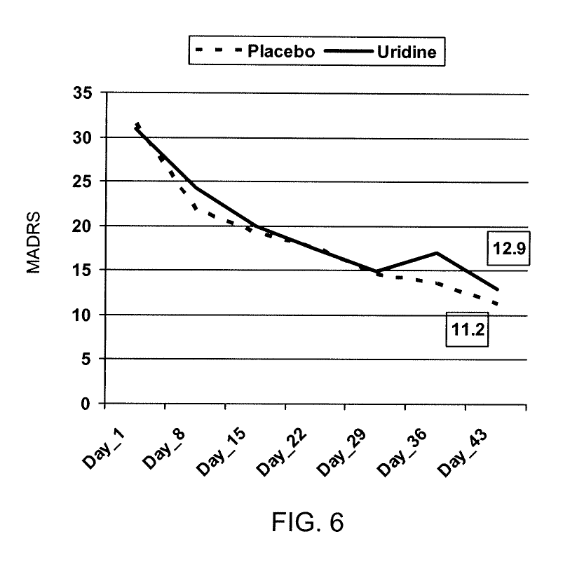

Similar results were seen at a threshold of five or ten episodes of depression (FIGS. 5-6. Patients with more than five episodes of depression over their entire life (n=50) demonstrated greater improvements in their symptoms of depression compared to patients with 5 or fewer episodes of depression over their entire life. From weeks 2-6 of treatment, the patients with more than 5 episodes of depression over their entire life who received uridine had an average improvement on MADRS of 5.5 points over placebo (p<0.001), compared to all patients in the study receiving RG2417 who had an average improvement on MADRS of 3.0 points over placebo (p=0.01).

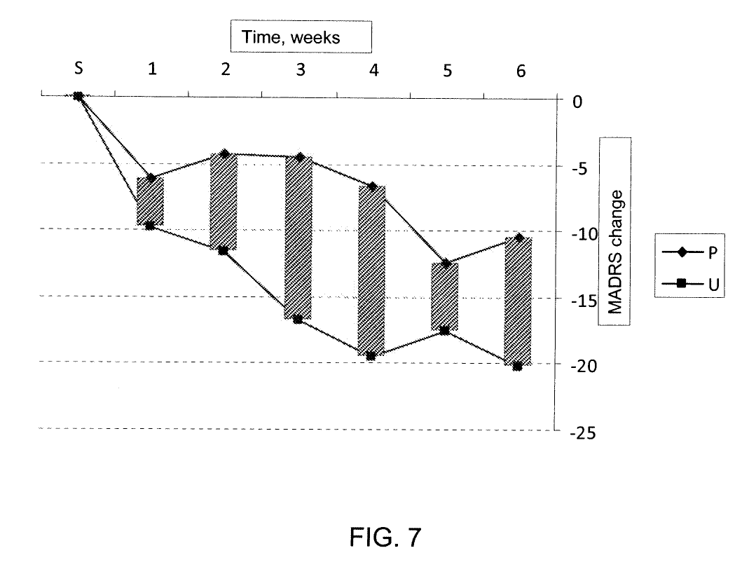

The response of bipolar I subjects with a history of 15 or more episodes of mania and/or hypomania were analyzed by comparison of the mean MADRS score to placebo during the trial. This criterion was intended to analyze the response in individuals with a more severe disease course. There was a large overlap with subjects in the depression severity subset, i.e., severe subjects tended to have many episodes of both depression and mania/hypomania. The overall response to treatment and the difference from placebo was greater for the subset of subjects with a greater history of mania and hypomania. The true difference in mean between groups was 9.46 at week 6, compared to 3.04 for the total score (FIG. 7). This difference was due to both a larger treatment response and a smaller placebo response than the total group.

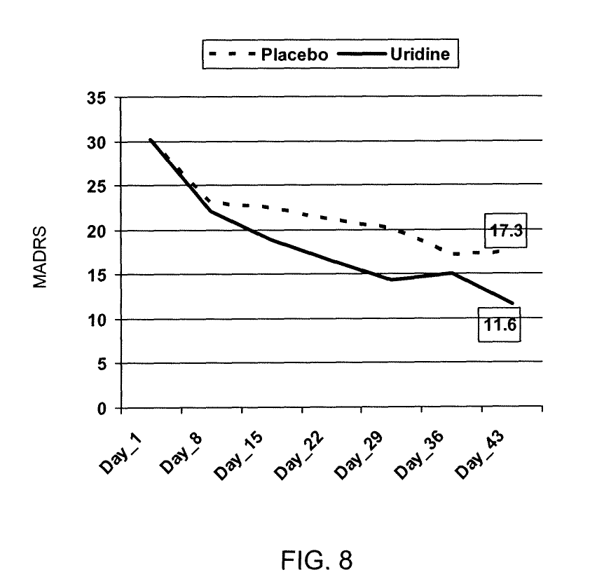

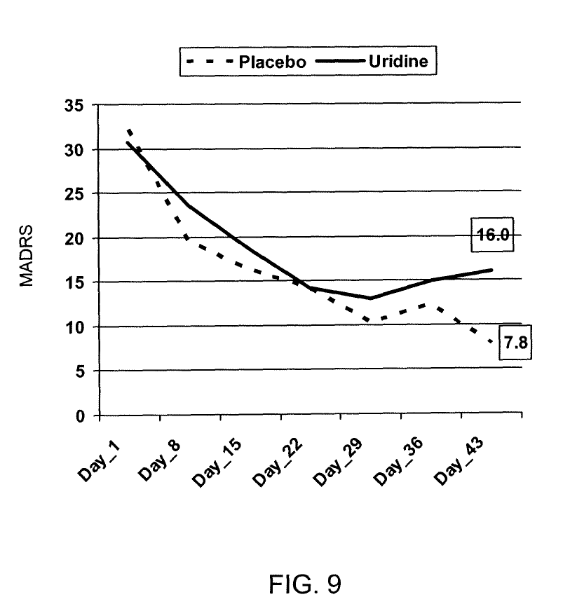

Similar results were obtained when the response of bipolar I subjects with a history of 2 or more or 5 or more episodes of mania were analyzed by comparison of the mean MADRS score to placebo during the trial. The overall response to treatment and the difference from placebo was also greater for these subsets of subjects. The true difference in mean between subjects with a history of 2 or more episodes of mania as compared to placebo was 5.7 at week 6, compared to 3.04 for the total score (FIG. 8). No positive response was observed in patients with a history of fewer than 2 episodes of mania (FIG. 9). The true difference in mean between subjects with a history of 5 or more episodes of mania as compared to placebo was 5.88 at week 6, compared to 3.04 for the total score (Table 3).

TABLE 3
Improvement in MADRS score for patients with 5 or
more manic episodes

| | |
|---|---|
| ≧5 Episodes | <5 Episodes |

| | |
|---|---|
| | Improvement |
| Day | (Uridine vs Placebo) |

| | | |
|---|---|---|
| 1 | 0.37 | −0.64 |
| 8 | 1.63 | −3.57 |
| 15 | 4.41 | −1.39 |
| 22 | 6.33 | −1.20 |
| 29 | 7.07 | −1.76 |
| 36 | 3.20 | −4.62 |
| 43 | 5.88 | −6.56 |

### Other Embodiments

A number of embodiments of the invention have been described. Nevertheless, it will be understood that various modifications may be made without departing from the spirit and scope of the invention. Accordingly, other embodiments are within the scope of the following claims.

1. A method of treating an individual diagnosed with bipolar disorder, the method comprising:
determining the number of manic episodes experienced by an individual exhibiting one or more symptoms of bipolar disorder; and
if the number of manic episodes is 2 or greater, administering to the individual an effective amount of a uridine composition.

2. The method of claim 1, wherein if the number of manic episodes is fewer than 2, a uridine composition is not administered to the individual.

3. The method of claim 1, wherein the effective amount of the uridine composition provides about 1 to 50 mg of a uridine composition/kg of body weight/day.

4. The method of claim 1, wherein the effective amount of the uridine composition provides about 3 to 10 mg of a uridine composition/kg of body weight/day.

5. The method of claim 1, wherein the individual is diagnosed under DSM-IV guidelines as having bipolar disorder and wherein the effective amount of the uridine composition comprises between 250 and 2000 mg of uridine composition/day.

6. The method of claim 1, wherein the composition is administered in two or three doses/day.

7. The method of claim 1, wherein the composition is uridine, triacetyl uridine, uridine monophosphate, uridine diphosphate, or uridine triphosphate.

8. A method of claim 1, wherein the effective amount of the uridine composition is administered orally.

9. A method of selecting an individual for treatment of bipolar disorder, the method comprising:
evaluating whether an individual exhibits one or more symptoms of bipolar disorder;
determining the number of manic episodes experienced by the individual; and
selecting the individual for treatment with a uridine composition if the number of manic episodes is 2 or greater.

10. The method of claim 9, wherein the method comprises selecting the individual for treatment with a uridine composition only if the number of manic episodes is 2 or greater.

11-12. (canceled)

13. A method of treating an individual diagnosed with bipolar disorder, the method comprising:
determining the number of manic episodes experienced by an individual exhibiting one or more symptoms of bipolar disorder;
determining the number of depressive episodes experienced by the individual; and
if the number of manic episodes is 2 or greater and the number of depressive episodes is 5 or greater, administering to the individual an effective amount of a uridine composition.

14. The method of claim 13, wherein if either the number of manic episodes is fewer than 2 or the number of depressive episodes is fewer than 5, a uridine composition is not administered to the individual.

15. A method of selecting an individual for treatment of bipolar disorder, the method comprising:
evaluating whether an individual exhibits one or more symptoms of bipolar disorder;
determining the number of manic episodes experienced by the individual;
determining the number of depressive episodes experienced by the individual; and
selecting the individual for treatment with a uridine composition if the number of manic episodes is 2 or greater and the number of depressive episodes is 5 or greater.

16-17. (canceled)

18. A method of evaluating a candidate treatment for bipolar disorder, the method comprising:
obtaining a population of individuals that exhibit one or more symptoms of bipolar disorder;
determining the total number of manic episodes experienced by each individual;
selecting a subpopulation of the population of individuals for whom the total number of manic episodes is 2 or greater; and
administering a candidate treatment for bipolar disorder to the subpopulation.

19. A method of evaluating a candidate treatment for bipolar disorder, the method comprising:
obtaining a population of individuals that exhibit one or more symptoms of bipolar disorder;
determining the total number of depressive episodes experienced by each individual;
selecting a subpopulation of the population of individuals for whom the total number of depressive episodes is 5 or greater; and
administering a candidate treatment for bipolar disorder to the subpopulation.

20. A method of evaluating a candidate treatment for bipolar disorder, the method comprising:
obtaining a population of individuals that exhibit one or more symptoms of bipolar disorder;
determining the total number of manic episodes experienced by each individual;
determining the total number of depressive episodes experienced by each individual;
selecting a subpopulation of the population of individuals for whom the total number of manic episodes is 2 or greater and the total number of depressive episodes is 5 or greater; and
administering a candidate treatment for bipolar disorder to the subpopulation.

21. The method of claim 9, further comprising administering to the individual an effective amount of a uridine composition.

22. The method of claim 15, further comprising administering to the individual an effective amount of a uridine composition.

23. The method of claim 2, wherein the effective amount of the uridine composition provides about 1 to 50 mg of a uridine composition/kg of body weight/day.

24. The method of claim 2, wherein the individual is diagnosed under DSM-IV guidelines as having bipolar disorder and wherein the effective amount of the uridine composition comprises between 250 and 2000 mg of uridine composition/day.
